(19) United States
(12) Patent Application Publication
(10) Pub. No.: US 2015/0204041 A1
(43) Pub. Date: Jul. 23, 2015

(54) TWO-TIER WIRELESS SOIL MEASUREMENT APPARATUS

(71) Applicant: Cheng-Hung Chang, Hsinchu (TW)

(72) Inventor: Cheng-Hung Chang, Hsinchu (TW)

(21) Appl. No.: 14/159,482

(22) Filed: Jan. 21, 2014

(51) Int. Cl. E02D 1/02; H04W 4/00; G01N 21/00; G01N 33/24; G01N 7/00; G01N 27/02; G01N 33/00; G01N 27/416; G05B 15/02; G01N 25/56

(52) U.S. Cl. CPC E02D 1/022; G05B 15/02; H04W 4/005; G01N 21/00; G01N 25/56; G01N 7/00; G01N 27/02; G01N 33/004; G01N 27/4167; G01N 33/24; E02D 1/027

(57) ABSTRACT

A two-tier wireless soil measurement apparatus is disclosed, including a top head and a plurality of sensors, wherein top head being placed above soil surface and the plurality of sensors being scattered under soil; each sensor including a sensor housing, first communication module, sensor unit and power module; the sensor unit sensing a soil condition and generating soil data representing the soil condition, the first communication module transmitting the soil data wirelessly to top head, and the power module providing power for sensor unit and first communication module; the top head including a first communication module, controller, second communication module and power module; the first communication module receiving soil data from first communication modules of sensors, the controller processing soil data, the second communication module transmitting the soil data wirelessly to a data station, and power module providing power to first communication module, controller and second communication module.

### FIELD OF THE INVENTION

The present invention generally relates to a two-tier wireless soil measurement apparatus for monitoring sub-surface soil conditions.

### BACKGROUND OF THE INVENTION

The wireless soil sensors are used to reduce water waste and water produce effective through continuous monitoring of the soil moisture level. The conventional wireless soil sensor system uses a probe buried into the soil. FIG. 1 shows a schematic view of a conventional wireless soil sensor probe. As shown in FIG. 1, a wireless soil sensor probe includes a top part 101 and a shaft part 102. The shaft part 102 is usually made in a tubular shape to house a plurality of sensors 1021, 1022, 1023, as shown in FIG. 1. The sensors 1021, 1022, 1023 monitor various soil conditions, such as, moisture, specific compounds, and so on, and pass the monitored data through a circuit to the top part 101. The top part 101 is exposed above the soil level 100 to transmit collected soil information to a data station 103.

The conventional wireless soil sensor probe shows certain practical disadvantages when deployed. For example, the deployment is often difficult. The shaft structure of the probe requires a vertical hole dug into the soil, which may encounter rocks or debris during digging. Also, it is not uncommon for some deployment to require the sensors requires buried deeper under the soil for more than 1 meter. The difficulty is high when considering a large-area field deployment of the wireless soil sensor probes.

### SUMMARY OF THE INVENTION

The present invention has been made to overcome the above-mentioned drawback of conventional wireless soil sensor system. The primary object of the present invention is to provide a wireless soil measurement apparatus that provides flexibility and ease for deployment.

An exemplary embodiment of the present invention discloses a two-tier wireless soil measurement apparatus, including a top head and a plurality of sensors, wherein the top head being placed on or above the ground and the plurality of sensors being scattered under the soil; each of the plurality of sensors including a sensor housing, a first communication module, a sensor unit and a power module; the sensor unit sensing a condition of the soil and generating soil data representing the soil condition, the first communication module transmitting the generated soil data to the top head, and the power module providing power for the operation of the sensor unit and the first communication module; the top head further including a first communication module, a controller, a second communication module and a power module; the first communication module receiving soil data from the first communication modules of the plurality of sensors, the controller processing the received soil data, the second communication module transmitting the processed soil data to a data station, and the power module providing power to the operation of the first communication module, the controller and the second communication module.

The foregoing and other objects, features, aspects and advantages of the present invention will become better understood from a careful reading of a detailed description provided herein below with appropriate reference to the accompanying drawings.

### BRIEF DESCRIPTION OF THE DRAWINGS

The present invention can be understood in more detail by reading the subsequent detailed description in conjunction with the examples and references made to the accompanying drawings, wherein:

FIG. 1 shows a schematic view of a conventional wireless soil sensor probe;

FIG. 2 shows a schematic view of a two-tier wireless soil measurement apparatus according to the invention;

FIG. 3 shows a schematic view of the structure of the top head according to the present invention;

FIG. 4 shows a schematic view of the structure of each of the plurality of sensor according to the present invention; and

FIG. 5 shows a diagram of the propagation loss of wireless signal for different frequencies according to the present invention.

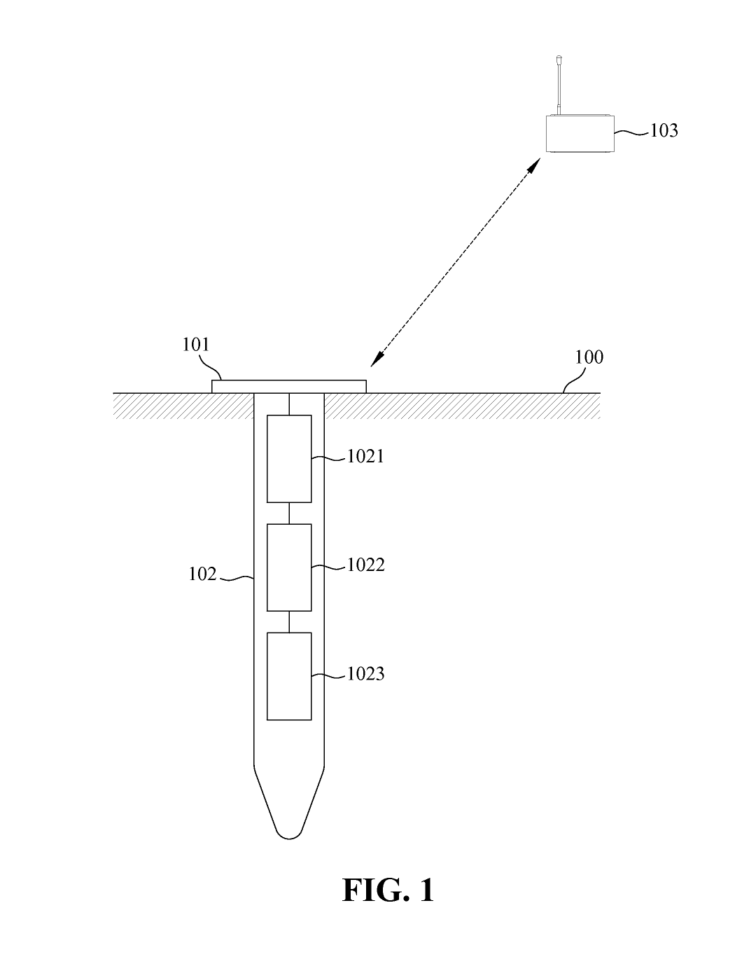

### DETAILED DESCRIPTION OF THE PREFERRED EMBODIMENTS

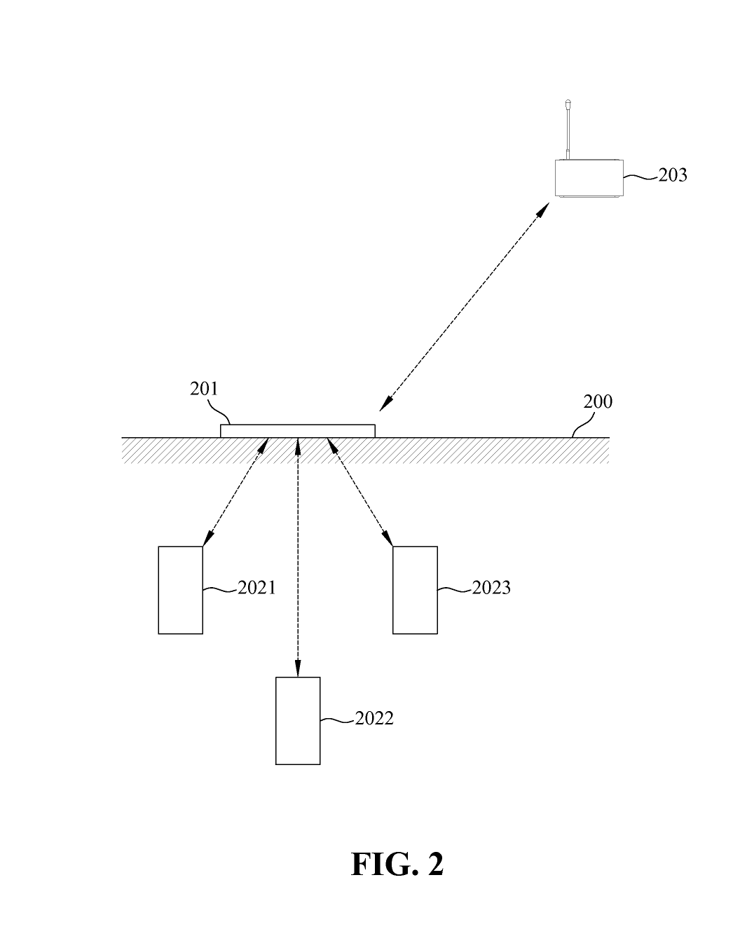

FIG. 2 shows a schematic view of a two-tier wireless soil measurement apparatus according to the invention. As shown in FIG. 2, the two-tier wireless soil measurement apparatus includes a top head 201 and a plurality of sensors 2021, 2022, 2023. The top head 201 is placed on or above the ground (i.e., soil surface) 200 for transmitting data collected from the plurality of sensors to a remote data station through a wireless means. The plurality of sensors is scattered under the soil. Each sensor is able to sense a soil condition, generate soil data representing the sensed soil condition, and transmit generated soil data to the top head through a wireless means.

It should be noted that the number of the sensors may vary and three sensors 2021, 2022, 2023 are shown in the present embodiment. In addition, the sensors 2021, 2022, 2023 may be buried under the soil at different depth and different vertical location. In addition, the soil condition monitored by each sensor 2021, 2022, 2023 may also be different. As such, the deployment of the sensors would allow higher flexibility to distribute different sensors at different depths and locations to monitor a wide range of soil conditions.

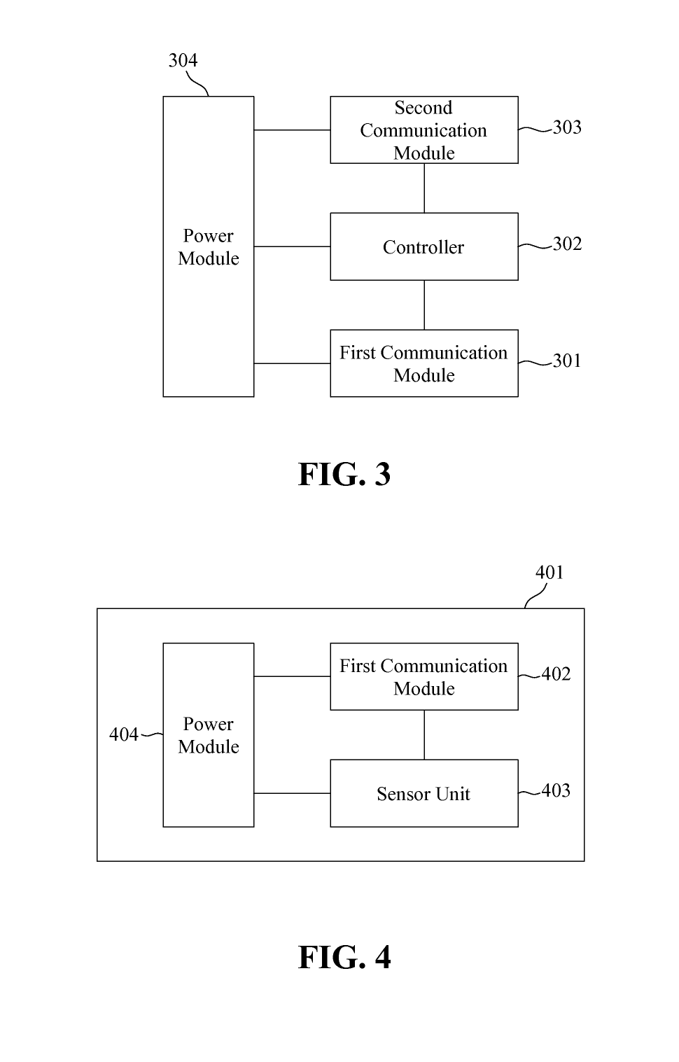

FIG. 3 shows a schematic view of the structure of the top head according to the present invention. As shown in FIG. 3, the top head further includes a first communication module 301, a controller 302, a second communication module 303 and a power module 304. The first communication module is for receiving soil data from each of the plurality of sensors 2021, 2022, 2023. The controller is connected to the first communication module for processing the received soil data and is connected to the second communication module for passing the processed soil data for transmitting. The second communication module is for transmitting the processed soil data to a data station (shown in FIG. 2), and the power module 304 is connected to the first communication module 301, the controller 302 and the second communication module 303 for providing power for the operations.

It should be noted that in a preferred embodiment, the first communication module and the second communication module are both wireless communication modules operating at different frequencies. This is because the propagation loss of the wireless signal is different for different operating frequency. For example, the operating frequency of the first communication module 301 is preferably ranging from 433 MHz to 1 GHz, and the operating frequency of the second communication module 303 is preferably between 900 MHz and 2.4 GHz. In the present embodiment, the first communication module 301 operates at 433 MHz and the second communication module operates at 2.4 GHz.

The top head 201 may further include one or more sensor units (not shown), connected to the controller for sensing various soil surface conditions, such as, air humidity level, air temperature, light level, CO₂ level, air pressure and so on. In addition, a GPS sensor or an accelerometer can also be included. The power module 304 may be a rechargeable power module. The additional ground level sensors and the aforementioned first communication module 301, controller 302, second communication module 303 and the power module 304 are all packed inside a housing case durable for soil and weather conditions.

FIG. 4 shows a schematic view of the structure of each of the plurality of sensor according to the present invention. As shown in FIG. 4, each of the plurality of sensors further includes a sensor housing 401, a first communication module 402, a sensor unit 403 and a power module 404. The sensor housing 401 is to provide housing to the aforementioned components, and is preferably made of metal, porous ceramic or plastic material in a tubular shape. The sensor unit 402 is disposed inside the sensor housing 401 for sensing a condition of the soil and generating soil data representing the soil condition. The first communication module 403 is also housed inside the sensor housing 401 and is connected to the sensor unit 402 for transmitting the generated soil data to the top head 201. The power module 404, also housed inside the sensor housing 401, is for providing power for the operation of the sensor unit 402 and the first communication module 403. Similarly, the power module 404 may be a rechargeable power module.

It should be noted that the first communication module 403 must operate at the same frequency as the first communication module 301 (in FIG. 3) of the top head 201 (in FIG. 2) to enable communication of soil data. Accordingly, the frequency range is preferably between 433 MHz and 1 GHz. In the present embodiment, the preferred operating frequency is 433 MHz.

Furthermore, in the present embodiment, the sensor unit 401 may be an accelerometer, a soil tension meter, a soil moisture sensor, a soil temperature sensor, a soil dissolved oxygen sensor, a soil pH level sensor, a soil conductivity sensor, a soil dielectric frequency sensor, or any combination of the above to monitor any necessary combination soil conditions.

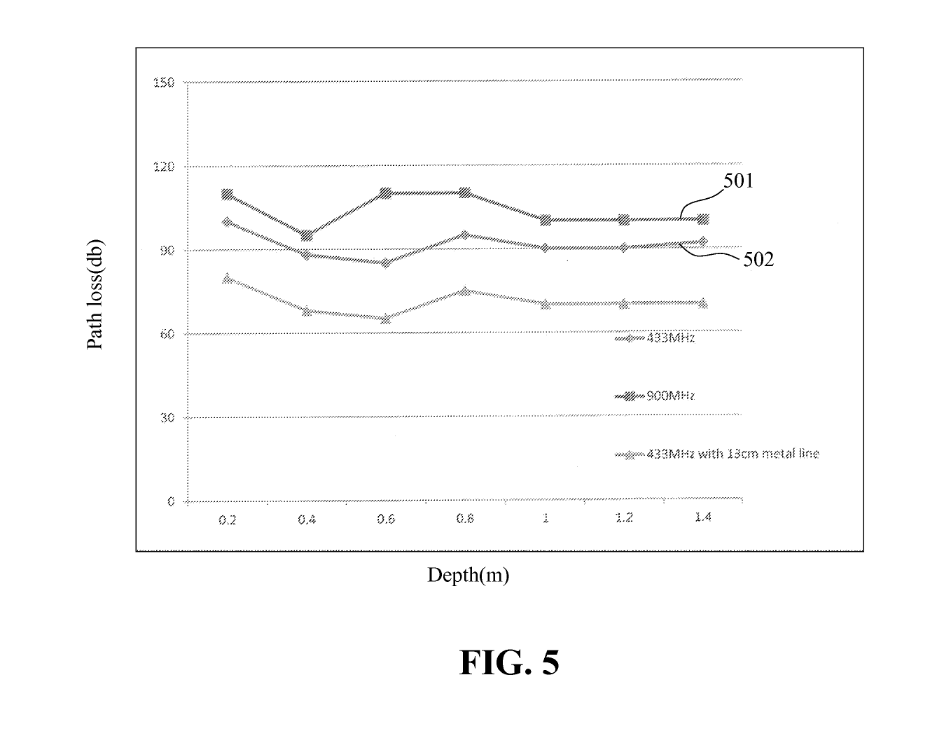

FIG. 5 shows a diagram of the propagation loss of wireless signal for different frequencies according to the present invention. As shown in FIG. 5, the line 501 indicates the signal loss at frequency 900 MHz versus depth of the soil, and the line 502 shows the signal loss at frequency 433 HMz. As seen, the signal loss is smaller at the frequency 433 MHz.

In summary, the two-tier wireless soil measurement apparatus of the present invention uses wireless communication to communicate soil data sensed and generated by the under soil sensors to the top head disposed above the soil so as to provide ease and flexibility of the deployment of the sensors to accommodate the underground condition.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A two-tier wireless soil measurement apparatus, comprising:
a top head, disposed above a soil surface for transmitting data to a data station through a wireless means; and
a plurality of sensors, disposed under soil for sensing soil conditions, generating soil data representing the sensed soil conditions, and transmitting generated soil conditions to the top head through a wireless means;
wherein each of the plurality of sensors comprising:
a sensor unit, for sensing a soil condition and generating soil data representing the soil condition;
a first communication module, connected to the sensor unit, for transmitting the soil data to the top head through a wireless means;
a power module, for providing power for the operation of the sensor unit and the first communication module; and
a sensor housing, for housing the sensor unit, the first communication module and the power module;
the top head further comprising:
a first communication module, for receiving data soil transmitted from the first communication module of each of the plurality of sensors;
a controller, connected to the first communication module for processing received soil data;
a second communication module, connected to the controller, for transmitting the processed soil data to the data station; and
a power module, connected to the first communication module, the controller and the second communication module for providing power.

2. The apparatus as claimed in claim 1, wherein both the first communication module of the top head and the first communication module of each of the plurality of sensors communicate data at a first frequency.

3. The apparatus as claimed in claim 2, wherein the first frequency is preferably between 433 MHz and 1 GHz.

4. The apparatus as claimed in claim 1, wherein the second communication module of the top head communicates data at a second frequency.

5. The apparatus as claimed in claim 4, wherein the second frequency is preferably between 900 MHz and 2.4 GHz.

6. The apparatus as claimed in claim 1, wherein the power module of each of the sensors is a wireless rechargeable power module.

7. The apparatus as claimed in claim 1, wherein the power module of top head is a wireless rechargeable power module.

8. The apparatus as claimed in claim 1, wherein the top head further comprises a housing case for housing the first communication module, the controller, the second communication module and the power module.

9. The apparatus as claimed in claim 8, wherein the top head further comprises one or more sensor units for sensing soil surface conditions, and the one or more sensor units are housed inside the housing case.

10. The apparatus as claimed in claim 9, wherein the one or more sensor units may be an air humidity level sensor, an air temperature sensor, a light level sensor, a CO₂ level sensor, an air pressure sensor, a GPS sensor, an accelerometer or any combination of the above.

11. The apparatus as claimed in claim 1, wherein the sensor housing is made of metal, porous ceramic, plastic or any combination of the above in a tubular shape.

12. The apparatus as claimed in claim 1, wherein sensor unit can be an accelerometer, a soil tension meter, a soil moisture sensor, a soil temperature sensor, a soil dissolved oxygen sensor, a soil pH level sensor, a soil conductivity sensor, a soil dielectric frequency sensor, or any combination of the above.
